(19) United States
(12) Patent Application Publication
(10) Pub. No.: US 2006/0200037 A1
(43) Pub. Date: Sep. 7, 2006

(54) System and method for selectively collecting exhaled air

(76) Inventor: Marianne Rita Falasco, Orange City, FL (US)

Correspondence Address: SACCO & ASSOCIATES, PA, P.O. BOX 30999, PALM BEACH GARDENS, FL, 33420-0999, US

(21) Appl. No.: 11/364,949

(22) Filed: Mar. 1, 2006

Related U.S. Application Data
(60) Provisional application No. 60/657,903, filed on Mar. 2, 2005.

(51) Int. Cl. A62B 18/08; A61B 5/08

(52) U.S. Cl. 600/538; 128/201.13; 128/905

(57) ABSTRACT

A system and method for selectively collecting exhaled air. The system includes a sensor 118 that can be configured for detecting a flow of exhaled air 130 from a person. A control system 120 can be responsive to the sensor 118. The control system 120 can be configured for generating a gate signal when at least one characteristic of the flow of exhaled air 130 satisfies one or more predetermined flow criteria. A valve 122 responsive to the gate signal can be configured for selectively gating only a predetermined portion of the flow of exhaled air to an air sample chamber 110.

### CROSS REFERENCE TO RELATED APPLICATIONS

This application claims benefit of U.S. Provisional application Ser. No. 60/657,903 filed on Mar. 2, 2005, which is incorporated by reference.

### BACKGROUND OF THE INVENTION

1. Statement of the Technical Field

The inventive arrangements relate generally to a system and method for selectively collecting exhaled air.

2. Description of the Related Art

Nitric oxide, also known as NO, is an important signaling molecule in the body of mammals, including humans. It is one of the few gaseous signaling molecules known. Because of its production in allergic reactions, there is research on using levels of exhaled nitric oxide, or eNO, to optimize the treatment of airway inflammatory disorders, such as asthma. Regular monitoring of a patient's eNO level can give both the medical caregiver and the patient valuable insight into the progression of the airway inflammation, which represents the principal cause of asthma. For example, a patient's eNO value can assist the physician in diagnosing asthma and in monitoring patient compliance and response to medical drug treatment. Moreover, it can alert physician and patient when there is an impending breakdown in asthma control.

There are several devices that have been developed to monitor eNO levels. For example, NIOX monitor, manufactured by Aerocrine, Inc. of Danderyd, Sweden is one such device used. The NIOX monitor includes a mask into which the person exhales. The exhaled air is then analyzed by a computer for the presence of NO. However, there are several disadvantages to such a system. First, the exhalation is not calibrated to measure a predetermined amount of air, resulting in a high level of error. Second, when monitoring for eNO, the device does not account for the amount of ammonia or ammonium gas in a person's exhalation. The presence of ammonia or ammonium gas in an initial portion exhaled air sample interferes with the detection of NO, thus creating an additional margin of error.

For the foregoing reasons, there is a need for an improved system and method for detecting eNO in a predetermined portion of exhaled air. The improved system should minimize the amount of ammonia and or ammonium gas particles that can be present in a sample portion of exhaled air. Such a system would provide a smaller margin of test error in the detection of eNO, resulting in improved diagnosis and treatment for airway inflammation disorders.

### SUMMARY OF THE INVENTION

The invention is directed to a system for selectively collecting exhaled air. The system can include a sensor configured for detecting a flow of exhaled air from a person. A control system can be provided that is responsive to the sensor. For example, the control system can be an electronic control circuit. The control circuit can be configured for generating a gate signal when at least one characteristic of the flow of exhaled air satisfies at least one predetermined criteria. The predetermined criteria can comprise a predetermined volume of exhaled air flowing continuously over a predetermined period of time. Alternatively, the predetermined criteria can comprise a predetermined mass flow rate of exhaled air. The characteristic of the flow of exhaled air can be selected to minimize the presence of ammonia in a predetermined portion of the flow of exhaled air.

A valve can be responsive to the gate signal. The valve can be configured for selectively gating only the predetermined portion of the flow of exhaled air to an air sample chamber. The valve can switch from a first position, which does not allow the predetermined portion of the flow of exhaled air to flow into the air sample chamber, to a second position, which does allow the predetermined portion of the flow of exhaled air to flow into the air sample chamber.

According to one embodiment of the invention, the air sample chamber can be configured for storing at least a part of the predetermined portion of the flow of exhaled air. Alternatively, or in addition to storing the air, the air sample chamber can be comprised of an adsorbent that is configured to adsorb certain types of chemicals or exhaled particulate matter. The adsorbent can be clinically evaluated at a later time to determine if certain chemicals or particles are present. The air sample chamber can also be comprised of a testing reagent. The testing reagent can be selected to respond to the presence of certain gasses or chemicals in the exhaled air. For example, the air sample chamber can be configured for detecting the presence of nitric oxide.

The system can also include at least one feedback mechanism that is responsive to the control system. The feedback mechanism can provide one or more indicators for communicating to a person that the air flow characteristic has been satisfied. Alternatively, or in addition to indicating that the air flow characteristic has been satisfied, the feedback mechanism can also indicate a degree to which the air flow characteristic has or has not been satisfied.

The invention also is directed towards a method for selectively collecting exhaled air. The method includes sensing a flow of exhaled air from a person. The method can also include selectively gating only a predetermined portion of the flow of exhaled air to an air sample chamber. According to one embodiment of the invention a gate signal can be generated when one characteristic of the flow of exhaled air satisfies one or more predetermined criteria. The predetermined criteria can be a predetermined volume of exhaled air flowing continuously over a predetermined period of time. According to another embodiment, the predetermined criteria can be a predetermined mass flow rate of the exhaled air. A predetermined portion of the flow of exhaled air can be selected to minimize its ammonia content.

A predetermined portion of the flow of exhaled air can be selectively gated to an air sample chamber. The method can include storing the predetermined portion of the flow of exhaled air in the air sample chamber for later testing. Alternatively, the method can include detecting the presence of nitric oxide in the predetermined portion of the flow of exhaled air. The method can further include the step of providing one or more indicators for communicating to a person that the characteristic has been satisfied.

### BRIEF DESCRIPTION OF THE DRAWINGS

FIG. 1 shows a perspective view of a system for selectively collecting exhaled air.

FIG. 2 shows a block diagram of the system of FIG. 1 when a valve is in a first position.

FIG. 3 shows a block diagram of the system of FIG. 1 when the valve is in a second position.

FIG. 4 shows a flow chart of a method for selectively collecting exhaled air.

### DETAILED DESCRIPTION OF THE PREFERRED EMBODIMENTS

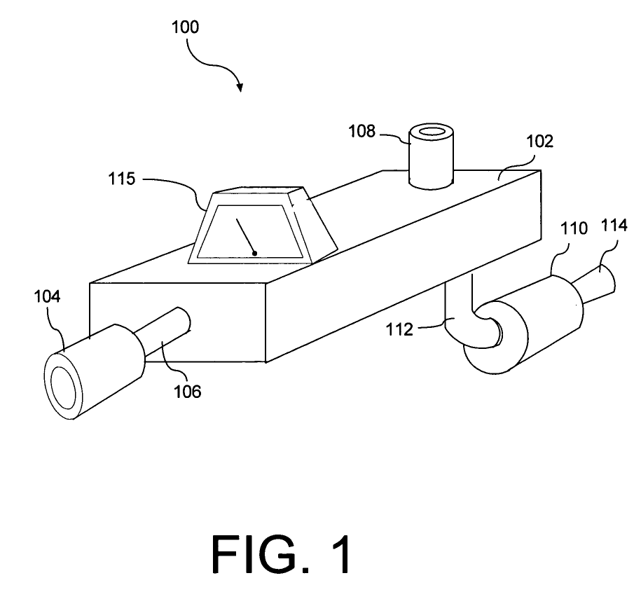

The present invention concerns a system 100 for selectively collecting exhaled air. Referring to FIG. 1, one embodiment of the system 100 is illustrated. The system 100 can include a housing 102. Housing 102 can be formed of any suitable material such as a rigid plastic or polymer shell. A mouthpiece 104 can be removably attached to an entry conduit 106 disposed on housing 102. A first exhaust vent 108 can be disposed on housing 102. An air sample chamber 110 can be attached to a sample conduit 112 disposed on the housing 102. For example, the air sample chamber 110 can be removably attached to the air sample conduit 112. In this regard, the air sample chamber 110 and the air sample conduit 112 can each include a corresponding fitting for removably attaching the two components together to form an airtight seal. According to one embodiment, a threaded fitting can be used. However the invention is not limited in this regard. A second exhaust vent 114 can be disposed on the air sample chamber 110. Furthermore, a feedback mechanism 115 can be disposed on the housing 102.

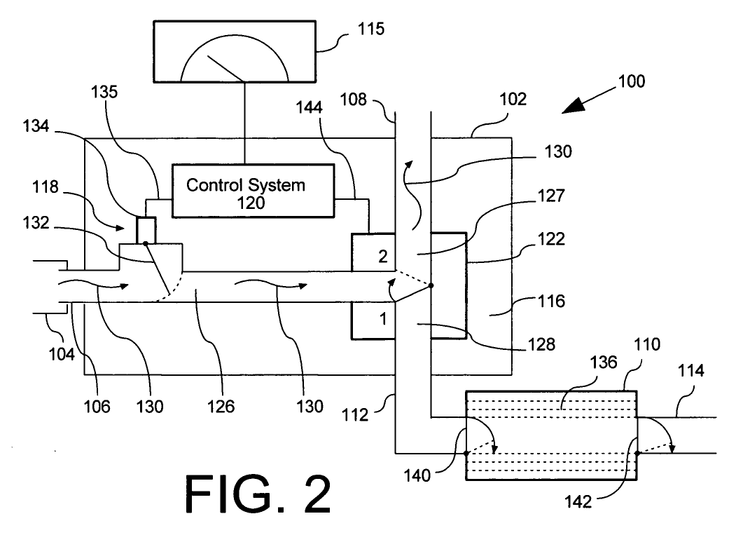

Referring now to FIG. 2, there is shown a block diagram that is useful for understanding the system 100. As illustrated therein, the housing 102 can include an internal cavity 116. The internal cavity 116 can be sized and shaped to at least partially enclose a sensor 118, a control system 120, and a valve 122. Moreover, the housing 102 can enclose various air conduits, such as a sensor air conduit 126, an exhaust air conduit 127, and a sample air conduit 128. It can be appreciated that the particular arrangement of the elements shown in the aforementioned embodiment is not critical to the invention and other configurations are possible.

It can be observed that the sensor 118 can be configured for detecting a flow of exhaled air 130 from a person. According to one embodiment shown in FIG. 2, the sensor 118 can include a vane 132 that is connected to a transducer 134. The transducer 134 can include any suitable device capable of converting the position of the vane 132 to a signal, such as an electronic signal. According to one embodiment, the transducer 134 can be a rotary potentiometer. However, the invention is not limited in this regard and other transducer devices can also be used. For example, an optical sensor, capacitance based sensor, or any other transducer device can be used for this purpose.

The vane 132 can be responsive to the flow of exhaled air 130 that enters the housing 102 via entry conduit 106 and flows through sensor air conduit 126. Depending on the relative position of the vane 132 caused by the flow of exhaled air 130 pushing against the vane 132, the transducer 134 will produce a different output. It can be appreciated that the invention is not limited in this regard and any suitable electrical or mechanical sensing mechanism can be used to detect the flow of exhaled air 130. For example, the sensor can alternatively be defined by a spring loaded displacement piston. As the flow of exhaled air comes in contact with the displacement piston, the displacement piston can push against the spring. The displacement produced can trigger the transmission of a control signal to the control system 120.

The control system 120 can be responsive to the sensor 118, and more particularly to the output 135 of the transducer 134.

According to one embodiment, the control system 120 can be electronic circuit. For example, the control system 120 can include a microcontroller or microprocessor. According to the embodiment shown in FIG. 2, the control system 120 can be configured to receive sensor data from transducer 134, which data can then be processed therein. The control system 120 can also be configured for generating a gate signal when one or more characteristics of the flow of exhaled air 130 satisfies one or more predetermined criteria.

One example of a predetermined criterion can include a predetermined volume of exhaled air flowing continuously over a predetermined period of time. This criterion can be generally referred to as the volumetric flow rate of a fluid. The predetermined period of time can be set to a range of about 2 seconds. However, the invention is not limited in this regard and other criteria can be used. For example, the predetermined criterion can be based upon a predetermined mass flow rate of the exhaled air. In order to employ mass flow rate as a criterion, the control system 120 can store the predetermined density of the exhaled air, as well as the predetermined cross-sectional area of the sensor air conduit 126 within a data store or memory unit (not shown) associated with the control system 120. Under this alternative criterion, the control system 120 can be responsive to the velocity of the exhaled air that is measured by sensor 118 over some period of time.

A valve 122 can be responsive to the gate signal 144 transmitted by the control system 120. More particularly, a position of the valve 122 can be automatically varied in response to the gate signal 144. Any suitable type of automatic valve can be used for this purpose. If the control system is an electronic system, the valve can be electronically actuated. For example, a solenoid or a stepper motor can be used to change the position of the valve 122. However, a pneumatically or hydraulically operated valve could also be used for this purpose. The particular type of valve selected can vary within the scope of the invention.

By changing the position of the valve 122 from a position 1 to a position 2, the valve 122 can selectively gate a predetermined portion of the flow of exhaled air toward first exhaust vent 108 or air sample conduit 112. However, the invention is not limited in this regard and other types of valve mechanisms can be used.

An air sample chamber 110 can be configured for storing at least part of the predetermined portion of the flow of exhaled air. In order to ensure that the sampled air is free of unwanted contaminants, the air sample chamber 110 can include a dual check valve system defined by a first check valve 140 and a second check valve 142. When the check valves 140, 142 are closed, they selectively contain the predetermined portion of the flow of exhaled air within the air sample chamber 110.

The air sample chamber can include an adsorbent 136 that is configured to adsorb selected chemicals and particulate matter that may be contained in exhaled air. Suitable commercially available adsorbents include, but are not limited to, activated carbon, silica gel, activated alumina, molecular sieve carbon, molecular sieve zeolites, silicalite, AIPO.sub.4, alumina, polystyrene, TENAX series, CARBOTRAP series, CARBOPACK series, CARBOXEN series, CARBOSEIVE series, PORAPAK series, SPHEROCARB series, Dow XUS series, and combinations thereof. Preferred low-pressure adsorbent combinations include, but are not limited to, TENAX TA and GR, CARBOTRAP, and Dow XUS565. According to one embodiment of the invention, the adsorbed exhaled air chemicals or particulate matter can be stored within the air sample chamber 110 for subsequent analysis and testing.

Alternatively, the air sample chamber 110 can be configured to directly detect and visually convey the presence of selected chemical compounds in the predetermined portion of the flow of exhaled air. For example, the air sample chamber can be configured to identify the presence of nitric oxide. According to this alternative embodiment, the air sample chamber 110 can include a testing reagent. The testing reagent can be defined by a dye deposited on a testing plate within the air sample chamber 110. The dye can produce a detectable spectral, transmission or reflectance response in the presence of selected chemicals or particulate matter. For example, the testing reagent can include, but is not limited to a calorimetric acidity indicator comprising phenylphthalein, bromthymol blue or methyl red. Other testing reagents that can be also used include Griess Reagents I and II.

The operational design of the system shall now be discussed. According to the embodiment of the invention shown in FIG. 2, when the valve 122 is in position 1, the flow of exhaled air 130 circulates through the sensor air conduit 126 and is then directed through exhaust conduit 127 and exits through the first exhaust vent 108. It can be observed that, with the valve 122 in position 1, there is no flow of exhaled air 130 circulating through the sample air conduit 128. In addition, the first and second check valves 140, 142 are closed, further serving as a redundancy in minimizing the amount of contaminated air sample that can enter the air sample chamber 110.

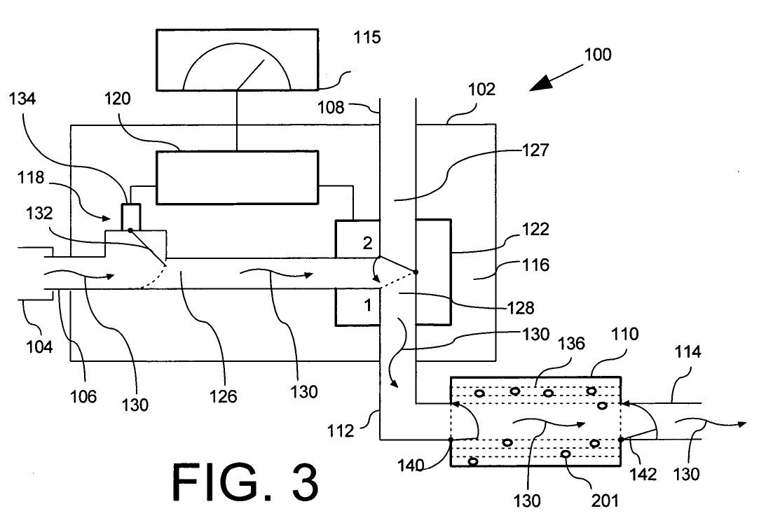

Referring now to FIG. 3, when the valve 122 is in position 2, the flow of exhaled air 130 circulates through the sensor air conduit 126 and is then diverted through the sample air conduit 128, of which a portion of the flow of exhaled air can be collected in the air sample chamber 110. When the valve is in position 2, the first and second check valves 140, 142 can open with the flow of exhaled air. The air entering the air sample chamber 110 can be retained in the sample chamber when the check valves 140, 142 close. If an adsorbent 136 is used, then the particulate matter 201 can be retained within the adsorbent 136 for later testing. According to yet another alternative, the air entering the sample chamber 110 can react with a reagent contained within the air sample chamber 110 to produce a visible test result.

One advantage of the foregoing invention is that it can selectively collect for testing only a predetermined portion of an exhaled breath of air. For example, the system 100 can divert a first portion of an exhaled flow of air to exhaust vent 108. A subsequent portion of a flow of the same exhaled breath can be collected. Selectively collecting a sample of air in this manner can be advantageous for certain types of tests. For example, in some instances, it can be desirable to minimize the ammonia content within a sample of exhaled air which is to be tested. Amonia content in exhaled air tends to be greatest at the beginning of the exhalation process and tends to be reduced toward the end of an exhaled breath. The system 100 can selectively collect for testing only the final or latter portion of an exhaled breath.

The system 100 can include a feedback mechanism 115 that is responsive to the control system 120. The feedback mechanism 115 can provide one or more indicators for communicating to a person that the characteristic of the flow of exhaled air 130 has been satisfied. For example, a graphic display such as a volumetric flow rate meter can be used as an indicator. However, the invention is not limited in this regard and other types of indicators can be used, such as an audible indicator. By using the feedback mechanism, a person can be informed when a sufficient quantity of breath has been expelled so as to begin collecting an air sample. For example, a short, low volume exhalation of breath may not satisfy the air sample collection requirements. In that case, the flow characteristic would not be satisfied. This information could be displayed to a user in the form of a metered air flow indication. Alternatively, any other indicator can be used for this purpose. For example, a light or audible alert can be sounded when the flow criteria have been satisfied for collecting a desired portion of an exhaled breath.

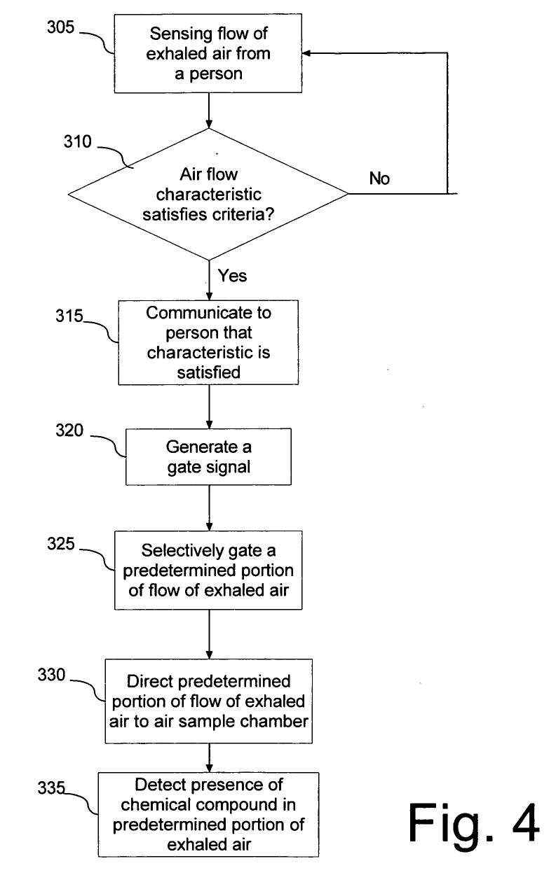

In FIG. 4, there is shown a flow chart 300 which is useful for understanding the method of the present invention. Beginning at step 305, a sensor can sense the flow of exhaled air from a person. In step 310, a determination is made whether the exhaled air satisfies one or more predetermined flow criteria. If the criteria is met, a feedback mechanism can optionally communicate to a person that an air flow characteristic has been satisfied, as shown in step 315. Continuing at step 320, a gate signal is generated and transmitted to a valve. Proceeding to step 325, the valve can selectively gate a predetermined portion of the flow of exhaled air. The predetermined portion of flow of exhaled air can then be stored in an air sample chamber 110, as shown in step 330. Lastly, the presence of selected chemical compounds in a predetermined portion of exhaled air can be detected, as shown in step 335. For example, step 335 can include detecting the presence of nitric oxide.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as described in the claims. Accordingly, reference should be made to the following claims, rather than to the foregoing specification, as indicating the scope of the invention.

What is claimed is:

1. A system for selectively collecting exhaled air comprising:
a sensor configured for detecting a flow of exhaled air from a person;
a control system responsive to said sensor, said control system configured for generating a gate signal when at least one characteristic of said flow of exhaled air satisfies at least one predetermined criteria; and
a valve responsive to said gate signal, said valve configured for selectively gating only a predetermined portion of said flow of exhaled air to an air sample chamber.

2. The system according to claim 1, wherein said predetermined criteria comprises a pre-determined volume of said exhaled air.

3. The system according to claim 2, wherein said predetermined volume of said exhaled air flows continuously over a predetermined period of time.

4. The system according to claim 1, wherein said predetermined criteria comprises a predetermined mass flow rate of said exhaled air.

5. The system according to claim 1, further comprising at least one feedback mechanism responsive to said control system, said feedback mechanism providing at least one indicator for communicating to a person that said characteristic has been satisfied.

6. The system according to claim 1, wherein said air sample chamber is configured for storing at least a part of said predetermined portion of said flow of exhaled air.

7. The system according to claim 6, wherein said air sample chamber is comprised of an adsorbent that is configured to adhere to exhaled air particulate matter.

8. The system according to claim 1, wherein said air sample chamber is configured for detecting the presence of nitric oxide.

9. The system according to claim 8, wherein said air sample chamber is further comprised of a testing reagent.

10. The system according to claim 1, wherein said valve switches from a first position, which does not allow said predetermined portion of said flow of exhaled air to flow into said air sample chamber, to a second position, which does allow said predetermined portion of said flow of exhaled air to flow into said air sample chamber when said gate signal is generated by said control system.

11. The system according to claim 1, wherein said at least one characteristic is selected to minimize the presence of ammonia in said predetermined portion of said flow of exhaled air.

12. A method for selectively collecting exhaled air comprising the steps of:
sensing a flow of exhaled air from a person;
generating a gate signal when one characteristic of said flow of exhaled air satisfies at least one predetermined criteria; and
selectively gating only a predetermined portion of said flow of exhaled air to an air sample chamber.

13. The method according to claim 12 wherein said predetermined criteria is a predetermined volume of exhaled air.

14. The method according to claim 13 wherein said predetermined volume of exhaled air flows continuously over a predetermined period of time.

15. The method according to claim 12 wherein said predetermined criteria is a predetermined mass flow rate of said exhaled air.

16. The method according to claim 12, further comprising the step of providing at least one indicator for communicating to a person that said characteristic has been satisfied.

17. The method according to claim 12, further comprising the step of storing said predetermined portion of said flow of exhaled air in an air sample chamber.

18. The method according to claim 17, further comprising the step of detecting for the presence of nitric oxide in said predetermined portion of said flow of exhaled air.

19. The method according to claim 12, further comprising selecting said predetermined portion of said flow of exhaled air to minimize its ammonia content.
